(19) United States
(12) Patent Application Publication
(10) Pub. No.: US 2026/0354680 A1
(43) Pub. Date: Nov. 12, 2026

(54) Determinining information about corneal water content

(71) Applicant: Aalto University Foundation sr, Aalto (FI)

(72) Inventors: Juha Ala-Laurinaho, Aalto (FI); Mariangela Baggio, Aalto (FI); Aleksi Tamminen, Aalto (FI); Zachary Taylor, Aalto (FI); Irina Nefedova, Aalto (FI); Roman Grigorev, Aalto (FI); Anton Chernenko, Aalto (FI)

(21) Appl. No.: 19/515,504

(22) PCT Filed: Sep. 4, 2024
(86) PCT No.: PCT/FI2024/050461

§ 371 Date: Mar. 2, 2026

(30) Foreign Application Priority Data
Sep. 7, 2023 (FI) 20237152

(51) Int. Cl. A61B 3/10

(52) U.S. Cl. CPC A61B 3/10

(57) ABSTRACT

According to an example aspect of the present disclosure, there is provided a measurement system (1) for determining information about corneal water content, wherein the system (1) is configured to illuminate a cornea system (40) using multiple Terahertz beams and receive backscattered versions of said multiple Terahertz beams, determine joint statistics of the backscattered versions of said multiple Terahertz beams and determine information about water content of the cornea (40) based on the joint statistics.

### FIELD

The present disclosure relates to corneal water content determination and more specifically, to a system and a method for determining information about corneal water gradient.

### BACKGROUND

Cornea is the transparent front part of the eye covering the iris, pupil, and anterior chamber. The cornea's optical properties are dependent on its water contents which is ~79% by mass. The water is distributed as a gradient in the thickness dimension where the top (anterior) is less hydrated than the bottom (posterior surface). The base layers (endothelium and Descemet's membrane) are responsible for maintaining Corneal Tissue Water Content, CTWC, by actively pumping out water naturally diffusing in from the aqueous humor. Determination of water content of the cornea would be beneficial at least for management and care of various diseases, and possibly for many other applications as well.

### SUMMARY

According to some aspects, there is provided the subject-matter of the independent claims. Some embodiments are defined in the dependent claims.

According to a first aspect of the present disclosure, there is provided a measurement system for determining information about corneal water content, wherein the system is configured to illuminate a cornea using multiple Terahertz beams and receive backscattered versions of said multiple Terahertz beams, determine joint statistics of the backscattered versions of said multiple Terahertz beams and determine information about water content of the cornea based on the joint statistics.

Example embodiments of the first aspect may comprise at least one feature from the following bulleted list or any combination of the following features:
wherein said information about water content of the cornea comprises at least one of a tissue water content gradient of the cornea or thickness of the cornea;
wherein the measurement system further comprises a Terahertz transmitter configured to illuminate the cornea using said multiple Terahertz beams;
wherein the measurement system further comprises a Terahertz receiver configured to receive said backscattered versions of said multiple Terahertz beams;
wherein the measurement system is further configured to illuminate the cornea using said multiple Terahertz beams with a frequency band between 100 GHz and 600 GHz, preferably between 140 GHz and 220 GHz, 220 GHz and 330 GHz or 330 GHz and 500 GHz;
wherein a number of said multiple Terahertz beams is between 20 and 30, preferably about 30;
wherein a number of said multiple Terahertz beams is sufficient for processing of material properties and deconvolving target location and confounders in the beam path;
wherein the measurement system is further configured to determine said joint statistics by calculating an average of said backscattered versions of said multiple Terahertz beams;
wherein the measurement system is further configured to determine said joint statistics by using particle swarm optimization on said backscattered versions of said multiple Terahertz beams.

According to a second aspect of the present disclosure, there is provided a method for determining information about corneal water content, comprising illuminating a cornea using multiple Terahertz beams and receiving backscattered versions of said multiple Terahertz beams, determining joint statistics based at least on the backscattered versions of said multiple Terahertz beams and determining information about water content of the cornea based on the joint statistics.

### BRIEF DESCRIPTION OF THE DRAWINGS

FIG. 1 illustrates a measurement system in accordance with at least some embodiments of the present disclosure;

FIG. 2 illustrates total water content in accordance with at least some embodiments of the present disclosure;

FIG. 3 illustrates bound water content in accordance with at least some embodiments of the present disclosure;

FIG. 4 illustrates an electromagnetic model of the Corneal phantom in accordance with at least some embodiments of the present disclosure;

FIG. 5 illustrates simulation results for a first frequency range in accordance with at least some embodiments of the present disclosure;

FIG. 6 illustrates simulation results for a second frequency range in accordance with at least some embodiments of the present disclosure;

FIG. 7 illustrates sensitivity analysis in three WR waveguides in accordance with at least some embodiments of the present disclosure;

FIG. 8 illustrates a flow diagram in accordance with at least some embodiments of the present disclosure.

### EMBODIMENTS

Embodiments of the present disclosure relate to corneal water content determination. More specifically, embodiments of the present disclosure provide a measurement system for determining information about corneal water content based on joint statistics which are further based at least on backscattered versions of multiple Terahertz beams. Moreover, simultaneous joint extraction of corneal posterior water content and corneal thickness is enabled by using a submillimeter wave reflectometry, wherein measurements are repeated many times in quick succession to enable definition and description of the joint statistics. Joint statistical features may be used to provide an estimate of corneal tissue water content gradient and thus, posterior corneal water content even when penetration of THz radiation to the posterior segment is negligible.

In some embodiments, a transceiver of the measurement system may illuminate the cornea with only one frequency at any given time. However, the frequency may be swept very fast so that, on a human time scale, it looks like all frequencies are being produced at once. Further, as the frequency is scanned, the shape of the beam, its source location, it's focus, etc. would not really change. Illumination may be thus considered as one beam, whose “color” changes very quickly or many beams where each frequency represents a different beam.

Diseases, such as Fuch's endothelial dystrophy, degrade the water maintenance function leading to increased Corneal Tissue Water Content, CTWC. Further, some immunologic responses, such as corneal graft rejection, are typically preceded by, or concomitant with, corneal edema. Later stage edema is detectable via visual assessment as the hyperhydrated cornea appears “milky” owing to the ~wavelength dependent scatter of engorged collagen fibers. However, treatment timed by detection via gross appearance often has limited efficacy. Early and accurate quantification of corneal edema is critical to the management and care of these ocular diseases and detrimental immune responses.

Diagnostic methods may restrict in vivo CTWC quantification to pachymetry, which is an ophthalmological test that measures the thickness of the cornea, and infers tissue hydration from thickness quantified via ultrasonic or optical backscatter measurements. However, the mapping from the pachymetry-measured Central Corneal Thickness, CCT, to corneal tissue hydration does not account for physiologic variation and returns erroneous, abnormal hydration values for corneal thicknesses off the nominal 580 μm. Hence, there is a need for non-contact, in vivo CTWC quantification.

Embodiments of the present disclosure therefore enable determining information about corneal water content using a THz system which is suitable, e.g., for mapping human cornea water content by coupling to the cornea's lossy longitudinal mode. When referenced to THz band wavelengths (λ~3-0.3 mm), the cornea presents as an optically smooth, spherical shell structure with radially varying dielectric properties arising from the water content gradient in the axial dimension. Furthermore, natural physiologic variation in the radius of curvature (Radius of Curvature, RoC, ~7.8 mm±0.5 mm), when referenced to THz band wavelengths, is negligible and thus, the cornea can be considered a perfect sphere with ~fixed RoC.

In case of the THz illumination of the cornea, the incident field may be modeled/assumed as a Gaussian beam in which the RoC of the wavefront matches the spherical surface at the optical axis. As the focused spot size of the incident field is less than the en face radius of the cornea, the electromagnetic properties of the cornea may be modelled with Effective Media Theories, EMT, to compute the effective permittivity at a given depth, and Stratified Media Theory, SMT, to capture the dielectric gradient and describe the aggregate, frequency dependent complex reflection coefficient. Optimization routines may be then applied to estimate corneal water content and thickness from backscattered THz illumination.

Embodiments of the present disclosure address more specifically at least two challenges. First, when the signal to noise drops, without improvements the fits would suffer from non-trivial uncertainty. Such uncertainty would result in a number of thickness, corneal posterior water, and corneal anterior water triplicates that all would yield the same goodness of fit. Second, the gradient and posterior water content may be so high that appreciable THz illumination energy does not make it to the posterior segment. In this case, the longitudinal mode content is negligible, the undulations in the spectra are suppressed, and even more uncertainty would be added to the measurement without improvements.

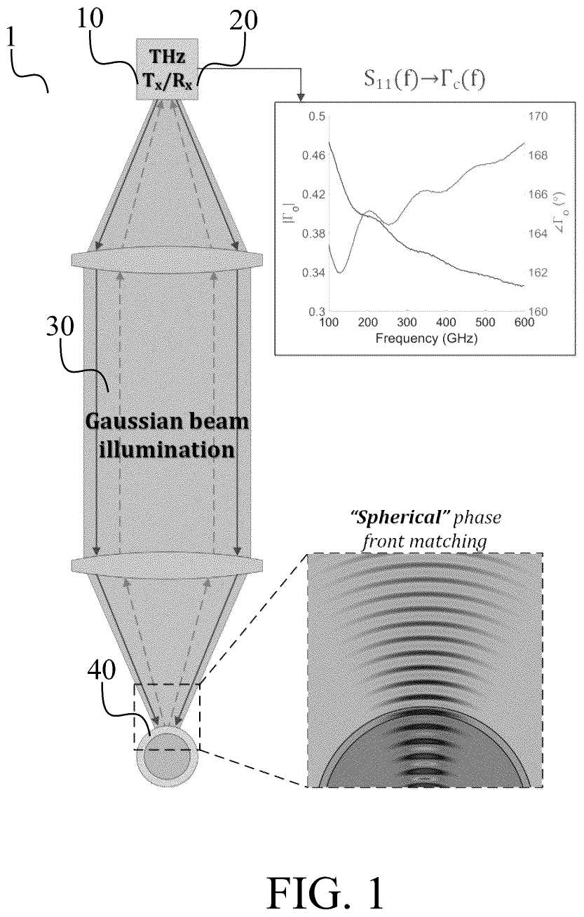

FIG. 1 illustrates a measurement system in accordance with at least some embodiments of the present disclosure. The measurement system (1) illustrated in FIG. 1 comprises a THz transmitter (10) and a THz receiver (20). In some embodiments, the measurement system (1) illustrated in FIG. 1 may comprise a Gaussian beam illumination device (30). Cornea is denoted by 40.

Custom optics, designed to fit to a specific antenna pattern, may be used to produce a close to ideal, laser like beam that matches the THz beam curvature to the corneal curvature. For example, a dual-reflector antenna, with two generatrix profiles configured to share a common ring focus, may be used. The primary reflector may feature a parabolic generatrix, while the electric field distribution at the focus may approximate a Bessel beam.

The THz transmitter (10) is configured to illuminate the cornea (40) using multiple THz beams and the THz receiver (20) is configured to receive backscattered versions of said multiple THz beams. The beams thus interact with the cornea (40) and underlying aqueous humor (water) with different interactions for different frequencies. These frequency varying interactions manifest as undulations and the reflection magnitude and phase across the illumination spectrum as demonstrated by in FIG. 1 (the box nearby the Gaussian beam illumination scheme). The thickness and corneal tissue water content gradient are encoded in these undulating spectra and fits to the spectra enable extraction of gradient and thickness.

The system (1) is therefore configured to illuminate the cornea (40) using said multiple THz beams and receive said backscattered versions of said multiple THz beams. The system (1) is further configured to determine joint statistics based at least on the backscattered versions of said multiple THz beams and determine information about water content of the cornea based on the joint statistics. In some embodiments, said information about water content of the cornea (40) may comprise a gradient of the water content of the cornea (40). The THz beams may be Gaussian beams.

In some embodiments, the system (1) may be further configured to illuminate the cornea (40) such that a curvature of the THz beams matches a curvature of the cornea (40). For example, the Gaussian beam illumination device (30) may be configured to illuminate the cornea (40) such that a curvature of the THz beam matches a curvature of the cornea (40), when the Gaussian beam illumination device (30) is incorporated in the system (1).

The system (1) may be further configured to illuminate the cornea using said multiple THz beams with a frequency band between 100 GHz and 600 GHz, preferably between 140 GHz and 220 GHz, 220 GHz and 330 GHz or 330 GHz and 500 GHz. A number of said multiple THz beams is between 20 and 30, preferably about 30.

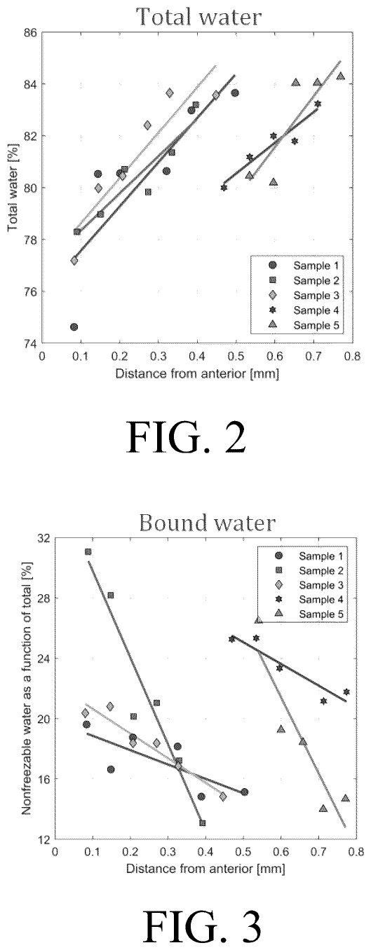

FIG. 2 illustrates total water content in accordance with at least some embodiments of the present disclosure. FIG. 3 illustrates bound water content in accordance with at least some embodiments of the present disclosure. FIG. 2 and FIG. 3 demonstrate data obtained in ex vivo animal eyes. FIG. 2 reports depth dependent total water content and FIG. 3 further delineates this to bound water percentage of total water. Some sample gradients would produce a very strong contrast. Others would produce a near perfect wave impedance match between the cornea (40) and aqueous humor, thus minimizing spectral undulations and maximizing measurement uncertainty. Embodiments of the present disclosure therefore leverage the measurement uncertainty weakness as a strength.

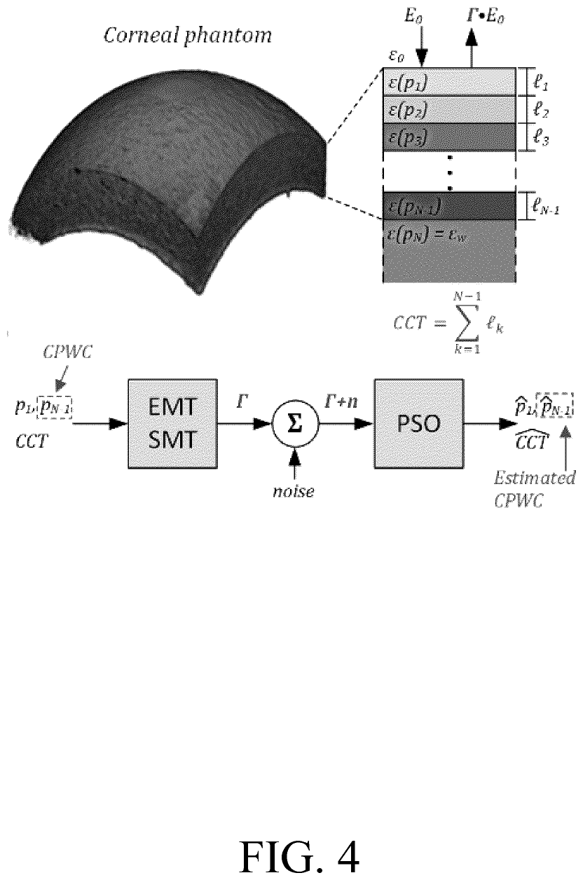

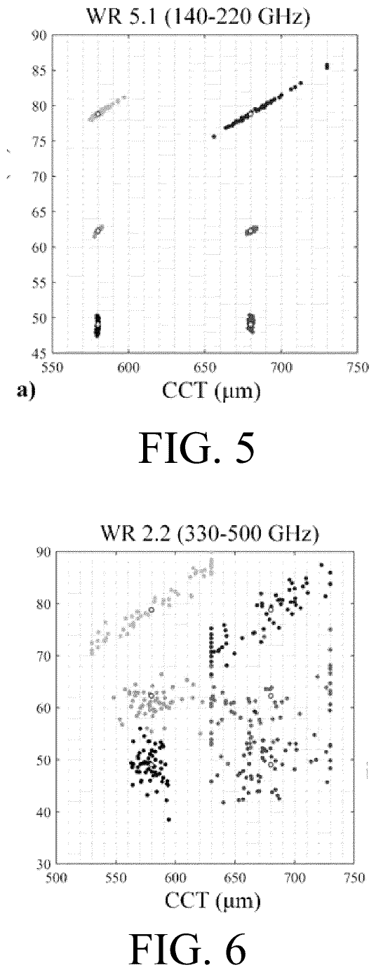

FIG. 4 illustrates an electromagnetic model of the Corneal phantom in accordance with at least some embodiments of the present disclosure. FIG. 5 illustrates simulation results for a first frequency range in accordance with at least some embodiments of the present disclosure. FIG. 6 illustrates simulation results for a second frequency range in accordance with at least some embodiments of the present disclosure.

FIGS. 4 to 6 illustrate a monte carlo simulation exercise. FIG. 4 shows the stratified media and effective media theory cornea forward model. The forward model is computed over a given illumination band, complex white gaussian noise is added, and then Particle Swarm Optimization, PSO, is used to solve the reverse problem. The system (1) may be thus further configured to determine said joint statistics by using PSO on said backscattered versions of said multiple THz beams.

The noise addition and reverse solution may be completed ~30 times for 6 different candidate posterior water, and corneal thickness pairs and the extracted posterior water content is plotted in FIG. 5 for 140-220 GHz and FIG. 6 for 330-500 GHz. For simplicity, the anterior water content was fixed. The horizontal axis is CCT and the vertical axis is Corneal Posterior Water Content, CPWC. The six CCT, CPWC noiseless pairs are represented by the small white circles and the noisy monte carlo iterations are distributed with colored markers about each noiseless point.

It is noted that very clear pattern arises in the 140-220 GHz band. The distribution in lower CPWC row is vertically oriented. The middle row is diagonally distributed with similar spans but different slopes. The top row feature shows very different span length but more similar slopes.

These features are still visible in the 330-500 GHz band but the results appear much noisier. This is not due to increased measurement noise but to reduced reflection from the posterior segment owing to the cornea's increased path loss at higher frequencies. While it is possible to assign one particular noisy point to a number or different noiseless points, consideration of a distribution of points both span and major and minor distribution axes, leads to a much more accurate determination of CPWC and CCT.

The uncertainty of one measurement may yield unacceptably large water content estimate errors. Characterization of uncertainty statistics using many measurements significantly reduces water content estimate errors. According to the embodiments of the present disclosure measurements are therefore not performed only once. Instead, measurements are performed many times and for example averaged. In such a case, wherein the system (1) may be further configured to determine said joint statistics by calculating an average of said backscattered versions of said multiple Terahertz beams. In some embodiments, measuring may be performed many times and the measurement uncertainty statistics quantified.

If it would be assumed either that (1) the CTWC gradient does not exist or (2) the gradient exists, is important, and reflection from the posterior segment, via significant remaining illumination energy at the posterior segment, is necessary for measurement. Assumption (1) is incorrect. Assumption (2) is a big risk as its very likely we cannot observe illumination field interaction with the posterior segment and thus cannot rely on lossy longitudinal modes for parameter extraction

Embodiments of the present disclosure substantially de-risk THz corneal sensing technology as it gives an alternative strategy of dealing with unexpectedly high path loss and/or unexpectedly high wave impedance matching between the anterior segment and aqueous humor.

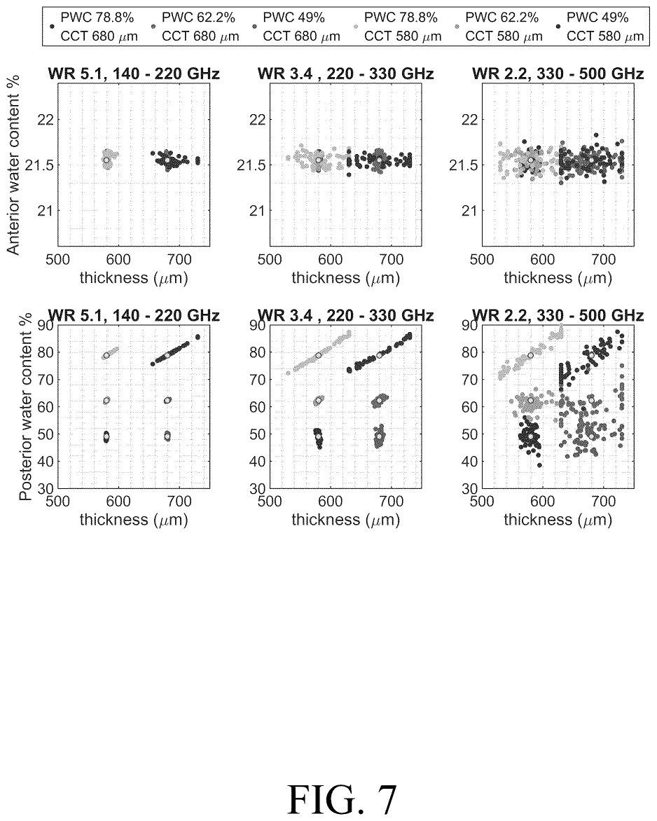

FIG. 7 illustrates sensitivity analysis in three WR waveguides in accordance with at least some embodiments of the present disclosure. More specifically, FIG. 7 illustrates noise sensitivity analysis in three WR waveguide (WR 5.1, WR 3.4, WR 2.2) bands with a SNR of 40 dB. FIG. 7 shows the extracted constituting parameters, i.e., Anterior Water Content, AWC, Posterior Water Content, PWC and CCT for six different configurations. Each configuration has a different shade of grey. Each column represents the same data set, but the first row focuses on the AWC vs thickness while the second on PWC vs thickness.

In FIG. 7, scatter plots represent an example of the joint distributions of estimated parameters. In the top row, the joint distributions describing the relationship between estimated front water content and thickness are illustrated. In the bottom row, the joint distributions describing the relationship between estimated back water content and thickness are illustrated. The distributions are dependent on the water content and thickness. Varying correlations can be observed. Joint statistics computed from these joint distributions may be exploited to form the basis of the measurement.

We simulated 18 different combinations of three corneal parameters for a given illumination band:
p_{front}→front (anterior) water content→(x1) value, Input triplicate;
p_{back}→back (posterior) water content→(x3) values, Input triplicate;
t_{cornea}—central corneal thickness→(x2) values, Input triplicate;
f→Illumination band→(x3) bands, Input Band.

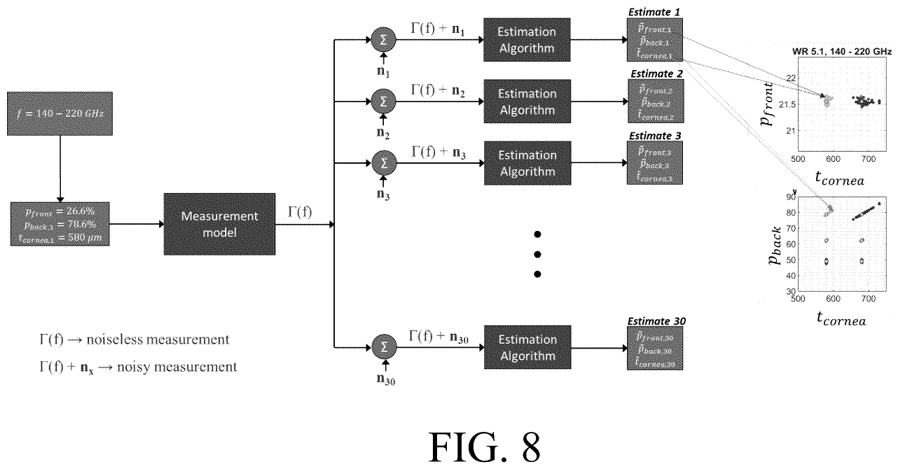

The parameters were fed into a model that predicts what a measurement would look like for a cornea described by a particular triplicate and a given operational band. This output is equivalent to a noiseless measurement. We calculated the noiseless measurement for all 6 combinations at all three bands for a total of 18 measurements. Then measurement noise was added to the noiseless measurements and the resulting noisy measurement signal was fed into an optimization algorithm that predicted in the input triplicate. To clarify, the output of the algorithm for a noiseless input is exactly the parameter triplicate input. The output of the algorithm for a noisy input is a triplicate that differs somewhat from the input triplicate. In other words, the best estimate of the input triplicate with a realistic input (noisy, non-ideal). This was done 30 times for each triplicate frequency band combination. An example is shown in FIG. 8, in the scatter plot.

FIG. 8 illustrates a flow diagram in accordance with at least some embodiments of the present disclosure. First, the frequency band may be selected. FIG. 8 illustrates the lowest band f=140-220 GHz. After that a parameter triplicate may be selected. In the example of FIG. 8, p_{front}=26.6%, p_{back}=78.6% and t_{cornea}=580 μm. The measurement model may be then used to generate noiseless measurement signal Γ(f). Random noise, mimicking measurement noise may be generated and indicated by the parameter n₁ and added to the noiseless data, thereby generating noisy data. Then an estimation algorithm may be used to estimate the operative parameters from the noisy data and we arrive at three estimates, denoted by the \tilde{p}_{front}, \tilde{p}_{back} and \tilde{t}_{cornea}. The estimated \tilde{p}_{front} is plotted vs the estimated \tilde{t}_{cornea} in the top scatter plot of FIG. 8 as indicated by the upper arrows. The f_{back} is plotted vs the estimated \tilde{t}_{cornea} in the bottom scatter plot of FIG. 8 as indicated by the lower arrows. The true parameters are superimposed as circles in both the top and bottom scatter plots.

This process may be then repeated 30 times to form the distribution of estimated parameters. In this way we can visualize the range of possible estimated parameters given the true parameters and a measurement subject to real-life measurement noise.

With reference to FIG. 7, the concern is that if the cornea is too lossy at the illumination frequency because it is (1) too hydrated and or (2) too thick, then only reflections from the front of the cornea would be acquired and no reflection from the back of the cornea. This would result in an estimate with substantial uncertainty. The uncertainty would be sufficient to make the measurement useless.

In case of the middle column of FIG. 7, which corresponds to the 220-330 GHz band, and considering the data points corresponding to ~62% free water at the back and 580 μm total thickness, with normal measurement noise the estimated front water content would range from ~21.4%-21.7%, yielding a total variation of ~0.3%. The estimated back water content would range from ~60%-64%, yielding a total variation of 4% which may or may not be sufficient.

Moreover, at least from data points corresponding to ~78% free water at the back and 680 μm total thickness, it may be derived that more water and increased thickness would lead to significant loss. With normal measurement noise the estimated front water content range would be approximately the same ~21.4%-21.7%, yielding a total variation of ~0.3%. However, the estimated back water content would range from ~73%-87%, yielding a total variation of ~14%, which would not be a sufficient fidelity.

Considering the scatter plots of the back water content, some dots show a moderate positive correlation between p_{back} and t_{cornea} with a limited span while some other dots show a very strong positive correlation with a very large span. According to embodiments of the present disclosure, multiple measurements are exploited to provide sufficient measurement accuracy. For example,
an ensemble of measurements may be taken, such as 30;
parameters estimation for each measurement may be performed;
the joint statistics of the estimated front water vs estimated thickness may be determined;
the joint statistics of the estimated back water vs estimated thickness may be determined;
potentially the known thickness may be used to reduce the distribution to one dimension;
characterization of the statistics of the parameters estimates from many measurements leads to sufficient measurement accuracy.

Concerning integrating, or averaging, for longer and improving the Signal to Noise Ratio, SNR, of a single measurement. Practically speaking, it would not be possible to average for long enough that the noise becomes negligible resulting in no uncertainty, because the patient and his/her eyes are always moving. Movement would show up in the data as noise or uncertainty that significantly larger than measurement noise. In other words, more averaging means worse signal.

If one would take many measurements as suggested above but instead of analyzing the uncertainty in the ensemble of extracted parameters, just add all the measurements together, the SNR would not be improved to drive down the uncertainty. The reason is that small differences in the physical location of the cornea between each measurement would result in averaging that is not coherent.

In some embodiments, Optical Coherence Tomography, OCT, may be used for two reasons. First, it may be ensured that the THz measurements of the cornea were obtained while the cornea is in the correct position. The OCT system may give this information in real-time with a depth resolution of ~9 μm; ~ 1/100 of a wavelength. Secondly, OCT may be used to a reference value for the cornea's thickness.

It is to be understood that the embodiments of the disclosure disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present disclosure. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present disclosure may be referred to herein along with alternatives for the various components thereof. It is understood that such embodiments, examples, and alternatives are not to be construed as de facto equivalents of one another, but are to be considered as separate and autonomous representations of the present disclosure.

Furthermore, the described features, structures, or characteristics may be combined in any suitable manner in one or more embodiments. In the preceding description, numerous specific details are provided, such as examples of lengths, widths, shapes, etc., to provide a thorough understanding of embodiments of the disclosure. One skilled in the relevant art will recognize, however, that the disclosure can be practiced without one or more of the specific details, or with other methods, components, materials, etc. In other instances, well-known structures, materials, or operations are not shown or described in detail to avoid obscuring aspects of the disclosure.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the disclosure. Accordingly, it is not intended that the disclosure be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

As used herein, “at least one of the following: <a list of two or more elements>” and “at least one of <a list of two or more elements>” and similar wording, where the list of two or more elements are joined by “and” or “or”, mean at least any one of the elements, or at least any two or more of the elements, or at least all the elements.

### INDUSTRIAL APPLICABILITY

At least some embodiments of the present disclosure find industrial application in corneal water content determination.

ACRONYMS LIST

| | |
|---|---|
| AWC | Anterior Water Content |
| CCT | Central Corneal Thickness |
| CPWC | Corneal Posterior Water Content |
| CTWC | Corneal Tissue Water Content |
| EMT | Effective Media Theories |
| OCT | Optical Coherence Tomography |
| PWC | Posterior Water Content |
| RoC | Radius of Curvature |
| SMT | Stratified Media Theory |
| SNR | Signal to Noise Ratio |

REFERENCE SIGNS LIST

| | |
|---|---|
| 1 | Measurement system |
| 10 | THz transmitter |
| 20 | THz receiver |
| 30 | Gaussian beam illumination device |
| 40 | Cornea |

1. A measurement system for determining information about corneal water content, wherein the system is configured to:
illuminate a cornea using multiple Terahertz beams and receive backscattered versions of said multiple Terahertz beams;
determine joint statistics of the backscattered versions of said multiple Terahertz beams; and
determine information about water content of the cornea based on the joint statistics.

2. The measurement system according to claim 1, wherein said information about water content of the cornea comprises at least one of a tissue water content gradient of the cornea or thickness of the cornea.

3. The measurement system according to claim 1, wherein the measurement system further comprises:
a Terahertz transmitter configured to illuminate the cornea using said multiple Terahertz beams.

4. The measurement system according to claim 1, wherein the measurement system further comprises:
a Terahertz receiver configured to receive said backscattered versions of said multiple Terahertz beams.

5. The measurement system according to claim 1, wherein the measurement system is further configured to:
illuminate the cornea using said multiple Terahertz beams with a frequency band between 100 GHz and 600 GHz.

6. The measurement system according to claim 1, wherein a number of said multiple Terahertz beams is between 20 and 30.

7. The measurement system according to claim 1, wherein a number of said multiple Terahertz beams is sufficient for processing of material properties and deconvolving target location and confounders in the beam path.

8. The measurement system according to claim 1, wherein the measurement system is further configured to:
determine said joint statistics by calculating an average of said backscattered versions of said multiple Terahertz beams.

9. The measurement system according to claim 1, wherein the measurement system is further configured to:
determine said joint statistics by using particle swarm optimization on said backscattered versions of said multiple Terahertz beams.

10. A method for determining information about corneal water content, comprising:
illuminating a cornea using multiple Terahertz beams and receiving backscattered versions of said multiple Terahertz beams;
determining joint statistics based at least on the backscattered versions of said multiple Terahertz beams; and
determining information about water content of the cornea based on the joint statistics.
